(19) United States
(12) Patent Application Publication
(10) Pub. No.: US 2017/0128530 A1
(43) Pub. Date: May 11, 2017

(54) MODIFIED PROCEDURE FOR CONTROLLED OVARIAN STIMULATION AND INTRAUTERINE INSEMINATION

(71) Applicant: CortControl, LLC, Santa Cruz, CA (US)

(72) Inventors: John Menear, Santa Cruz, CA (US); Joseph Ramaekers, Aptos, CA (US)

(21) Appl. No.: 15/410,502

(22) Filed: Jan. 19, 2017

Related U.S. Application Data
(63) Continuation-in-part of application No. 15/265,155, filed on Sep. 14, 2016.
Application No. 15/265,155 is a continuation of application No. 14/121,670, filed on Oct. 6, 2014, now Pat. No. 9,463,218.
Application No. 14/121,670 is a continuation-in-part of application No. 13/999,875, filed on Mar. 29, 2014, now abandoned.
Application No. 13/999,875 is a continuation-in-part of application No. 13/729,923, filed on Dec. 28, 2012, now Pat. No. 9,125,874.
Application No. 13/729,923 is a continuation-in-part of application No. 12/325,199, filed on Nov. 30, 2008, now Pat. No. 8,357,663.
(60) Provisional application No. 62/388,204, filed on Jan. 20, 2016.
(60) Provisional application No. 61/964,100, filed on Dec. 24, 2013.

(51) Int. Cl. A61K 38/17; A61K 31/716; A61K 35/744; A23L 33/00; A61K 9/00; A23L 33/16; A23L 33/105; A23L 33/135; A61K 36/07; A61K 33/00

(52) U.S. Cl. CPC A61K 38/1709; A61K 36/07; A61K 31/716; A61K 35/744; A61K 33/00; A61K 9/0056; A23L 33/16; A23L 33/105; A23L 33/135; A23L 33/40; A23V 2002/00

(57) ABSTRACT

A method of using a transfer factor formulation to improve the success rate of a human COS or COS/IUI procedure. The transfer factor formulation consists of at least transfer factor, and may also include lactic acid generating bacteria, and/or glucans. The transfer factor formulation, administered correctly, improves the probability of achieving conception via COS/IUI. Both men and women can receive transfer factor formulation. Dosage amounts are adjusted for body weight. Consumption frequency may be adjusted in response to hormonal measurements. Typically, consumption of the formulation begins three to 100 days before a planned COS/IUI procedure.

### CROSS REFERENCE TO RELATED APPLICATIONS

This application is a continuation-in-part of U.S. patent application Ser. No. 15/265,155, filed Sep. 14, 2016, which is a continuation of U.S. patent application Ser. No. 14/121,670, filed Oct. 6, 2014, which is a continuation-in-part of U.S. patent application Ser. No. 13/999,875, filed Mar. 29, 2014, which is a continuation-in-part of U.S. patent application Ser. No. 13/729,923, filed Dec. 28, 2012 and issued Sep. 8, 2015 as U.S. Pat. No. 9,125,874, which is a continuation-in-part of U.S. patent application Ser. No. 12/325,199, filed Nov. 30, 2008 and issued Jan. 22, 2013 as U.S. Pat. No. 8,357,663. U.S. patent application Ser. No. 13/999,875 claims priority to U.S. Provisional Patent Application No. 61/964,100, filed Dec. 24, 2013. This application also claims the priority to U.S. Provisional Patent Application No. 62/388,204, filed Jan. 20, 2016. All of the above patents and applications are specifically incorporated herein by reference in their entirety.

### TECHNICAL FIELD

Embodiments herein relate to methods of controlled ovarian stimulation and intrauterine insemination, and specifically for methods of controlled ovarian stimulation and intrauterine insemination augmented with transfer factor formulations.

### BACKGROUND

In the United States, the number of women ages 15-44 with impaired fecundity (impaired ability to get pregnant or carry a baby to term) is about 6.7 million. Multiple fertility clinics exist to help women with fertility issues. A well-established clinical fertility procedure is controlled ovarian stimulation (COS) and intrauterine insemination (IUI). COS/IUI is usually explored before considering in-vitro fertilization (IVF), and is far less expensive. The COS/IUI procedure is a two-step process, where the COS step is used to chemically release a healthy egg at a known time while IUI is used to fertilize the egg at the optimal time. IUI includes insemination by a clinician at a fertility clinic or timed intercourse. Typically COS is initiated using an ovulation stimulatory drug such as clomifene (marketed under the tradename Clomid™) or Letrozole (marketed under the tradename Femara™) to induce ovulation followed by IUI with donor sperm. Currently, a single COS/IUI cycle has a 6-8% probability of success. While the cost of COS/IUI is considerably less than IVF, raising the COS/IUI success probability would benefit infertile couples, and importantly, those that cannot afford IVF.

### BRIEF DESCRIPTION OF THE DRAWINGS

Embodiments will be readily understood by the following detailed description in conjunction with the accompanying drawings and the appended claims. Embodiments are illustrated by way of example and not by way of limitation in the figures of the accompanying drawings.

FIG. 1 is a flow diagram of a COS/IUI test protocol modified with a transfer factor formulation, in accordance with embodiments disclosed herein.

FIG. 2 is a timeline taken from a standard COS/IUI test protocol.

FIG. 3 is a timeline taken from a COS/IUI test protocol modified with a transfer factor formulation, in accordance with embodiment disclosed herein.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

Unless otherwise noted, technical terms are used according to conventional usage. Although methods and materials similar or equivalent to those described herein can be used in the practice or testing of the present disclosure, suitable methods and materials are described below. All publications, patent applications, patents, and other references mentioned herein are incorporated by reference in their entirety. Specifically U.S. Pat. Nos. 9,463,218; 9,125,874; and 8,357,663 and U.S. patent application Ser. Nos. 13/999,875; 13/843,581; and 13/718,319 are specially incorporated herein by reference in their entirety. In case of conflict, the present specification, including explanations of terms, will control.

Definitions of common terms in molecular biology can be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710); and other similar references.

Suitable methods and materials for the practice or testing of this disclosure are described below. Such methods and materials are illustrative only and are not intended to be limiting. Other methods and materials similar or equivalent to those described herein can be used. For example, conventional methods well known in the art to which this disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999. In addition, the materials, methods, and examples are illustrative only and not intended to be limiting.

### I. Introduction

Infertility is a growing problem in the United States. Approximately 6.7 million women in the U.S. between the ages of 15 and 44 experience an impaired ability to get pregnant or carry a baby to term. This equates to about 11% of women in this age group. In addition, approximately 7.4 million women report that they have used infertility services (see, for example, cdc.gov/nchs/fastats/infertility). Couples who have failed to conceive by chance alone after 12 months, and who have not been diagnosed with abnormalities known to affect fertility, are classified as having “unexplained infertility.” Up to 37% of infertile couples are estimated to be in this category. The most common treatment for these couples is controlled ovarian stimulation (COS) followed by intrauterine insemination (IUI) with the partner's sperm, or timed intercourse.

Different drug regimens have been used for COS+IUI/insemination protocols, each with different success rates and risk for multiple pregnancies. Recently, a large randomized clinical trial comparing three COS+IUI regimens found that protocols using the drug clomiphene resulted in pregnancy rates of approximately 28% after 4 COS cycles. However, undergoing COS is expensive and often not covered by insurance. Women who do not conceive after several COS cycles and who wish to continue fertility treatments are faced with more invasive and costly procedures with even lower success rates, for example in-vitro fertilization (IVF). Taken together, infertility is a costly and stressful problem for a large number of couples in the U.S.

The consuming public now understands that foods possess more than basic nutrition (protein, carbohydrate, fat, etc.). For example, many consumers agree that certain foods have health benefits that go beyond basic nutrition and that may reduce the risk of disease or other health concerns. Furthermore, a large percentage of consumers believe that foods can replace the use of drugs in certain situations. The Federal Drug Administration (FDA) has acknowledged this trend with the advent of the relatively new category of products termed “Medical Foods,” see, for example 21 CFR 101.9(j)(8). These Medical Foods should be administered or monitored by a doctor, nutritionist, nurse, medical technician or equivalent health care professional.

Disclosed herein is a food-based modification of COS/IUI fertility treatment using a medical food based formulation that includes transfer factor. By using food-based augmentation of COS/IUI fertility treatment, more infertile couples can be helped at reasonable cost, while avoiding expensive IVF treatments. Furthermore, the transfer factor formulations are foods, which may allow a subject consuming such foods to avoid drug side effects that may be present with more traditional fertility treatments. The disclosed methods improve the outcome of controlled ovarian stimulation (COS) and intra-uterine insemination (IUI), such as COS/IUI in a human subject, for example a human female subject. One of the outcomes that is observed from coupling a transfer factor formulation to COS/IUI is an improvement in egg quality and number in females, see Example 1. In addition, when transfer factor formulations are administered to male subjects, the result is higher sperm counts. Both conditions set the stage for improved physiology and improved outcomes going into COS/IUI. While not being bound by theory, it is believed that the biochemical mechanism by which the improved COS/IUI outcome occurs at least partly through a reduction in cortisol, such as measured cortisol, immune system improvement, and improved endocrine balance.

The disclosed methods employ the administration of transfer factor formulations, such as medical foods containing transfer factor, to improve the outcome of a COS/IUI procedure. In some embodiments, the transfer factor formulations include glucans and/or lactic acid generating bacteria. In some embodiments, the methods and compositions disclosed herein improve the COS/IUI of infertile women, men, and/or couples, for example those that have been unable to conceive over a period of 12 months via unprotected intercourse.

### II. Transfer Factor Compositions

As disclosed herein, transfer factor, even administered alone, can improve COS/IUI outcomes. Thus, in embodiments, a medical food for use in improving COS/IUI outcomes includes transfer factor. Such a composition is termed herein a transfer factor composition. In a certain embodiments, the transfer factor present in the transfer factor composition is derived from colostrum or egg.

Colostrum is a form of milk produced by the mammary glands of mammals (including humans) in late pregnancy and contains transfer factor and antibodies to protect the newborn against disease. Although transfer factor can be extracted from colostrum, it is noteworthy that transfer factor and colostrum are not the same. Roughly 1000 grams of colostrum is required to produce 0.8 grams of transfer factor. Thus, transfer factor represents a small percentage (perhaps 0.5 to 1 part-per-thousand) of colostrum. Hence, substitution of colostrum for transfer factor will lack sufficient transfer factor, and thus, substitution of colostrum for transfer factor is not recommended. While the mechanism of transfer factor on fertility is not known, it is believed to be related to an immune modulating effect of transfer factor.

Transfer factor is produced by leucocytes and lymphocytes. Transfer factor comprises small water soluble polypeptides of about 44 amino acids that stimulate or transfer cell mediated immunity from one individual to another. Transfer factors are small molecules (3500-10000 molecular weight), which in embodiments, contain RNA bases attached to short chain amino acids. These molecules were named for their potential to transfer immunity from one individual to another without exposure to the pathogen. For example, bovine transfer factor prepared from the colostrum of cows previously immunized for laryngotracheitis (respiratory disease) initiated antigen-specific cell-mediated immunity in non-immune T cells of specific pathogen-free chickens. In addition, it is also reported that transfer factor decreases cortisol in animal models, which may also have a beneficial effect on immune function.

Cow colostrum is typically a major source of transfer factor. Alternative sources of transfer factor include avian transfer factor, ova transfer factor, and colostrum from goats, pigs, horses or humans among others. In embodiments, combinations of transfer factors from multiple sources may be used in transfer factor formulations. In certain embodiments, the transfer factor is derived from a bovine, such as cattle source. Mixed colostrum from pooled cattle herds is preferable to colostrum from a single cattle herd because different herds may be exposed to different antigens, and pooled colostrum reflects a combined antigen exposure. Thus, pooling may be used to assure wide scope benefit. A superior grade of transfer factor is obtained by pooling sources of colostrum and/or egg-derived sources. Transfer factor is commercially available, and generally recognized as safe. Without limitation, the transfer factor compositions may be solid, such as a bar or liquid, such as a drink, for example similar to a protein powder drink. In certain embodiments of transfer factor formulations, substantially purified transfer factor has an average molecular weight of less than 10,000 Daltons. The properties, characteristics and processes for obtaining transfer factor or transfer factors used in the instant application are discussed in U.S. Pat. Nos. 4,816,563; 5,080,895; 5,840,700; 5,883,224; and 6,468,534, the contents of which are specifically incorporated herein by reference.

Lactic acid generating bacteria is a useful addition to a transfer factor containing medical food, and is generally recognized as safe. Lactic acid generating bacteria support digestion and brain health. Lactic acid generating bacteria provide healthful effects that are found in non-pasteurized sauerkraut and cod liver oil. Within the intestinal tract, lactic acid generating bacteria are beneficial. It has been estimated that 80% of human health depends on beneficial intestinal bacteria. A human body becomes weakened by poor digestion, and COS/IUI will reflect that weakening. Lactic acid generating bacteria help strengthen the body via improved digestion. Thus, in certain embodiments, a transfer factor formulation includes lactic acid generating bacteria.

Glucans (polysaccharides) are known to support the immune system. When combined with transfer factor, a synergy is created. The combined effect on health has been demonstrated in animals. In some embodiments, a transfer factor formulation includes glucans, such as glucans derived from mushrooms. In some embodiments, the glucans may be present as natural or hybrid mushrooms. In some embodiments, a formulation for COS/IUI enhancement includes transfer factor, glucans, and lactic acid generating bacteria. Transfer factor, glucans and lactic acid generating bacteria may be consumed at different times. For example within a one week period the transfer factor, glucans and lactic acid generating bacteria may be consumed.

Formulations for COS/IUI may be augmented with additives other than transfer factor, lactic acid generating bacteria, and glucans. The use of additives remains within the disclosure scope, providing that transfer factor is present. In embodiments, the transfer factor formulations include, such as are augmented with, additional additives that may further improve COS/IUI outcome. In some embodiments, an additive present in the transfer factor formulations includes one or more of minerals (such as essential minerals), probiotics, prebiotics, dimethyl glycine, ascorbic acid, Vitamin A, Vitamin D3, Vitamin E, Vitamin B1, Vitamin B2, Vitamin B12, electrolyte mixtures, dipotassium phosphate, potassium chloride, magnesium sulfate, calcium pantothenate, antioxidants, amino acids, nutraceuticals, inositol hexaphosphate (Ip6), mannans, olive leaf extract, and phytosterols.

In certain embodiments, the transfer factor formulation includes mannans, such as mannans derived from Aloe vera. In certain embodiments, mannans are consumed separately from the transfer factor formulation. In certain embodiments, the transfer factor formulation includes phytosterols, such as phytosterols derived from soybean. In certain embodiments, phytosterols are consumed separately from the transfer factor formulation.

In certain embodiments, the transfer factor formulation includes probiotics, such as, but not limited to, B. subtlis, B. longum, B. thermophilium, B. coagulans, E. faecium, and S. cerevisia, L. casei, L. plantarum, Pediococcus acidilacticii, Kluyveromyces marxianus fragillis, and combinations thereof. In certain embodiments, probiotics, such as one or more of B. subtlis, B. longum, B. thermophilium, B. coagulans, E. faecium, and S. cerevisia, L. casei, L. plantarum, Pediococcus acidilacticii, Kluyveromyces marxianus fragillis, are consumed separately from the transfer factor formulation.

A particularly useful additive is potassium, for example ionic potassium. The USDA, Agricultural Research Service, has reported that mean potassium intake by adults in the United States was approximately 2600 mg per day; far below the recommended 4700 mg per day. Potassium deficiency is associated with hypertension, which is undesirable during COS/IUI. In addition, hypertension complicates an estimated 5-10% of pregnancies. Therefore, potassium supplementation supports healthier reproductive function. The benefits of appropriate potassium intake accrue from potassium supplementation 1) before COS/IUI, 2) during COS/IUI, and 3) during gestation. In certain embodiments, the transfer factor formulation includes potassium. In certain embodiments, potassium is consumed separately from the transfer factor formulation.

The list of additives above is not exhaustive. Thus, in addition to the additives described above, in embodiments, the transfer factor formulation may also include one or more of the following: carrier proteins, such as serum albumin; buffering agents, such as sodium acetate; fillers, such as microcrystalline cellulose, lactose, corn and other starches; binding agents; sweeteners and other flavoring agents; coloring agents; and polyethylene glycol. Additional additives known in the art may be used in a variety of formulations.

### III. Methods of Improving COS/IUI Outcomes

Animal testing indicates that consuming a transfer factor formulation with COS improves birth rates, relative to COS without transfer factor formulation. Birth rates were particularly high with a combination of transfer factor, glucans, and lactic acid generating bacteria. Thus, the associated measurements support the expectation of higher birth rates. For example, cattle studies indicate that superior quality and quantity of eggs are collected from the donor cow after dietary inclusion of transfer factor, glucans, and lactic acid generating bacteria. This result was confirmed by microscopy, where egg size and morphology were metrics.

Aspects of the present disclosure relate to methods of improving the conception success rate of a human female subject undergoing a controlled ovarian stimulation and intrauterine insemination (COS/IUI) procedure. The disclosed methods include administering to the female subject, prior to COS, a medical food formulation comprising an effective amount of transfer factor, and continuing administration of the medical food formulation within the period between COS and IUI, wherein the COS/IUI conception rate obtained with the formulation is greater than the COS/IUI conception rate obtained without the formulation. In some embodiments, the methods include selecting a female subject for treatment, for example a female subject that has not been able to conceive for about 12 months or longer. In some embodiments, the female subject is tested for tubular blockage prior to COS and a female subject having tubular blockage is not selected, for example, excluded from treatment. In some embodiments, the female subject's male partner is tested for sperm count prior to COS and/or prior to IUI, for example, to exclude the couple from treatment, such as when the male's sperm count is low or nonexistent. In such a situation, the couple would not likely benefit from the treatment, or would likely benefit from the use of donor sperm.

Typically the dosages of transfer factor formulations are adjusted for the subject's weight. Humans typically respond to dosage levels that mimic animal levels, thus animal studies can be used to establish human dosages. However, recommended human treatment durations and human frequency of consumption may differ from other mammals. Optimal human treatments allow adjustment during the COS/IUI procedure. In embodiments, an effective amount of transfer factor is selected based on the female subject's body weight. In certain embodiments, the effective amount of transfer factor is from about 0.05 mg to about 50 mg per pound of human body weight, such as from about 0.05 mg to about 0.1, about 0.1 mg to about 0.25, about 0.25 mg to about 0.5, about 0.5 mg to about 1.0, about 1.0 mg to about 2.0, about 2.0 mg to about 3.0, about 3.0 mg to about 4.0, about 4.0 mg to about 5.0, about 5.0 mg to about 6.0, about 6.0 mg to about 7.0, about 7.0 mg to about 8.0, about 8.0 mg to about 9.0, about 9.0 mg to about 10.0, about 10.0 mg to about 15.0, about 15.0 mg to about 20.0, about 20.0 mg to about 25.0, about 25.0 mg to about 30.0, about 30.0 mg to about 40.0, about 40.0 mg to about 50.0, and the like.

Administering a transfer factor formulation typically occurs prior to the COS/IUI procedure. In some cases, administration may extend into the COS/IUI procedure and into the gestation period. Consumption 100 days prior to planned COS/IUI is a preferred scenario. The reasons are: (1) it takes about 80-90 days for a fully mature egg to develop, and (2) it takes about 72 days for a fully mature sperm to develop. By consuming a transfer factor formulation 100 days prior to COS, both the egg and sperm are fully developed in the best nutritional environment. Of course, consumption for shorter periods remains useful. People are different and pursue COS/IUI at varying levels of health. Consumption for 3 days prior to COS may be useful for some healthy couples. Because transfer factor formulations are inherently safe, consumption more than 100 days prior to COS/IUI is appropriate where practical. In some embodiments, administration begins from 3-100 days before the COS procedure, such as 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days, or in some cases even greater than 100 days, before the COS procedure. Furthermore, when consumption of transfer factor formulation begins well before the anticipated date for COS/IUI, COS/IUI may become unnecessary. This is another reason to begin consumption of the transfer factor formulation approximately 100 days before planned COS/IUI. In embodiments, the method further includes extending the administration until pregnancy is confirmed. In embodiments, the methods further include extending the administration through the entire pregnancy. In embodiments, the methods further include repeating the COS/IUI procedure with the administration of the medical food formulation. In embodiments, the female subject's consumption of the medical food formulation ceases on or before the day of IUI. In embodiments, ultrasound is used to confirm pregnancy.

In embodiments, the methods further include administering an effective amount of glucans or polysaccharides to the female, male, or both subjects. In embodiments, the glucans are derived from natural or hybrid mushrooms. In embodiments, an effective amount of glucans is about 0.1 mg to about 10.0 mg per pound of human body weight, such as about 0.1 mg to about 0.25, about 0.25 mg to about 0.5, about 0.5 mg to about 1.0, about 1.0 mg to about 2.0, about 2.0 mg to about 3.0, about 3.0 mg to about 4.0, about 4.0 mg to about 5.0, about 5.0 mg to about 6.0, about 6.0 mg to about 7.0, about 7.0 mg to about 8.0, about 8.0 mg to about 9.0, and about 9.0 mg to about 10.0 per pound of human body weight.

In embodiments, the methods further include administering an effective amount of lactic acid generating bacteria to the female, male, or both subjects. In embodiments, an effective amount of lactic acid generating bacteria is about 0.47 mg to 10 mg per pound of human body weight, such as about 0.47 mg to about 0.5, about 0.5 mg to about 1.0, about 1.0 mg to about 2.0, about 2.0 mg to about 3.0, about 3.0 mg to about 4.0, about 4.0 mg to about 5.0, about 5.0 mg to about 6.0, about 6.0 mg to about 7.0, about 7.0 mg to about 8.0, about 8.0 mg to about 9.0, and about 9.0 mg to about 10.0 per pound of human body weight.

Variation occurs among people, and variation occurs for a given person over time. Although typical proportions can be borrowed from animal testing, proportions may be modified to best serve each individual. Relative to livestock, human variation is wider. Recommended weight ranges allow for proportion modification. Dosages can change with time as hormone levels fluctuate. Periodic measurement of hormone levels followed by dosage adjustment is appropriate before and during COS/IUI. Treatment times are more flexible for human COS/IUI than for animal husbandry procedures. These pre-COS/IUI consumption periods are guidelines rather than rigid rules. Sometimes, practical reasons over-ride and short consumption times are used. Animal studies suggest that even 3 days of consumption is useful. Frequency of feeding differs between animals and humans. For livestock, feeding frequency is operationally fixed. Changing the feeding frequency affects the rancher's profit. Humans are different; for example, in human subjects several small transfer factor formulation portions per day—rather than 1 large portion per day—is not difficult to arrange. The primary human guideline is effectiveness, not economics. Consumption frequency may vary between five times per day and once per week. Further, each fertility food portion throughout the day does not have to be the same. For example, a subject with sleep issues may want to purposely time consumption to maximize sleep.

In embodiments, the transfer factor formulations are administered between five times per day and once per week, for example once every week, twice a week, three times a week, four times a week, five times a week, six times a week, daily, twice daily, three times daily, four times daily, five times daily, or even more.

In some embodiments, transfer factor, lactic acid generating bacteria, and glucans are taken together. Component separation and consumption at different times are within the scope of this disclosure. Thus, in some embodiments, transfer factor, lactic acid generating bacteria, and glucans are taken at different times during the day or week. In some embodiments, the transfer factor and lactic acid generating bacteria are separated and consumed at different times within a one week period.

Although much emphasis is placed on female subjects, male reproductive health and function are also improved by a transfer factor formulation comprising (1) transfer factor and lactic acid generating bacteria or (2) transfer factor, lactic acid generating bacteria, and glucans. An improved male contribution results from quantity or quality of sperm produced by the male. Roughly one-third of infertility problems originate with the male. In one dramatic animal study, a bull with negligible sperm count produced an above-average sperm count after 90 days of consuming transfer factor, lactic acid generating bacteria, and glucans. In embodiments, the methods further include administering to a male reproductive partner of the female subject the medical food formulation comprising the effective amount of transfer factor, wherein the effective amount is based on the body weight of the male. In one embodiment, human males begin treatment with transfer factor formulation 100 days before planned COS or COS/IUI. This period is usually sufficient to address low sperm count and low sperm quality that are 2 (or more) standard deviations below the male United States population mean. In another embodiment, human males begin treatment with the transfer factor formulation 30 to 60 days before COS/IUI. This period is considered sufficient to address sperm count and sperm quality that are 1 standard deviation below the United States population mean. The highest probability of success arises from both male and female consuming the transfer factor formulation prior to COS/IUI.

The method of using transfer factor formulation prior to COS/IUI may have some or all of the following steps:

(1) determine reference points by measuring hormonal levels and sperm count before and during COS/IUI,

(2) select the proportion of transfer factor, lactic acid generating bacteria, and glucans that comprise the mixture,

(3) choose the dosage level for the mixture,

(4) select a feeding frequency between five times per day and once per week,

(4) begin consumption before planned COS/IUI,

(5) periodically measure hormone levels and adjust food dosage, frequency, or both,

(6) continue consumption until IUI is performed,

(7) continue consumption until a stable pregnancy is achieved, or

(8) continue consumption throughout the gestation period.

### EXAMPLES

### Example 1

This Example describes an exemplary protocol for augmentation of COS/IUI with a transfer factor formulation.

The transfer factor formulation is dispensed with instructions for both female and male partner to begin daily consumption on day 1 of the female's next menstrual cycle. For example: the subject has an estimated menstrual cycle of 28 days during which she and her partner will take transfer factor formulation by mouth daily. The controlled ovarian stimulation protocol is initiated on day 2 or 3 of the following menstrual cycle. IUI is initiated on day 11-17. The male partner may continue taking transfer factor formulation until pregnancy is confirmed at 12 weeks. If pregnancy does not occur after the first COS cycle, the male partner may continue transfer factor formulation until, and throughout, the female partner's second COS cycle and for up to 12 additional weeks following the first COS cycle. Total days of transfer factor formulation consumption for the male partner could be 260+/−14 days over 2 COS/IUI cycles.

During an initial clinical visit, physicians perform a basic physical exam, medical history and demographics assessment, along with measures of body weight, height, etc., as per standard of care for COS fertility treatment. At this time, a blood sample is drawn from the female subject for anti-mullerian hormone (AMH) assessment of ovarian reserve. Typically an AMH level>1 to participate is desired. The male subject provides a semen sample for analysis in order to determine sperm count and motility. Sperm of 10 million and motility>20% is desired. Height and body weight is measured, and medical history and demographic information will be collected. Couples will be given the transfer factor formulation supplement at one of the baseline visits, and instructed to begin taking daily doses of transfer factor formulation on the first day of the female partner's next menstrual period and to continue doses up to, and throughout the COS+IUI/insemination protocol. Transfer factor formulation daily doses for both partners will begin on the first day of menses for the female partner, and continue until timing noted below.

After consuming a transfer factor formulation for one complete menstrual cycle, eligible subjects return on day 2-3 of the next menstrual cycle to begin the controlled ovarian stimulation cycle (COS). The procedure is variable among subjects and physicians. In examples, subjects may undergo transvaginal sonography and bloodwork for LH, FSH, E2, and progesterone. The purpose of these assessments is to check for antral follicle count and endometrial thickness and to rule out the presence of ovarian cysts. Blood may also be collected to ensure that female subjects are in the early follicular phase. For all COS subjects, Clomid® (clomiphene citrate, a SERM) or Femara® (letrozole, an aromatase inhibitor) pills are provided and the female subject will be instructed to start the COS treatment on day 2-3 of the menstrual cycle and continue at home for 5 days. On the second day after the COS medications are completed (day 10-12 of the menstrual cycle), subjects may return to the clinic for a second visit for transvaginal sonography to count follicles, measure follicle size, and measure LH, E2, and P4. If desired, the couple may opt to have ovulation triggered with HCG (human chorionic gonadotropin) on day 14-15 of the menstrual cycle when there are 1 to 4 follicles >15 mm in size and E2 is between 200 and 1000. Thirty-six hours post-HCG, or as a course of “timed intercourse” determination by the couple, insemination will be performed. On the day of IUI or timed intercourse decision, a fourth overnight urine sample from both partners and a second semen sample is provided. The entire COS+IUI cycle takes approximately 12-15 days and both male and female subjects continue to consume the transfer factor formulation daily until IUI, at which point, the female partner may discontinue.

Around 14-16 days after insemination (˜day 60 after transfer factor formulation initiation), the female subject returns for pregnancy testing via a blood test. If a subject is pregnant, then they will return for an ultrasound at 6 weeks to confirm pregnancy. An additional ultrasound may be performed at 12 weeks to reconfirm pregnancy as standard of care. Medical records are accessed approximately 10 months later to determine live birth outcomes.

If a subject is not pregnant, she and her male partner may continue the transfer factor formulation treatment and start another COS+IUI cycle. Transfer factor formulation may be consumed for multiple cycles. The female partner will discontinue transfer factor formulation treatment on the day of IUI for each attempted COS cycle. The male partner may continue taking the transfer factor formulation until pregnancy is confirmed at 12 weeks. If pregnancy does not occur after the first COS cycle, the male partner may continue transfer factor formulation treatment until, and throughout, a further COS cycle.

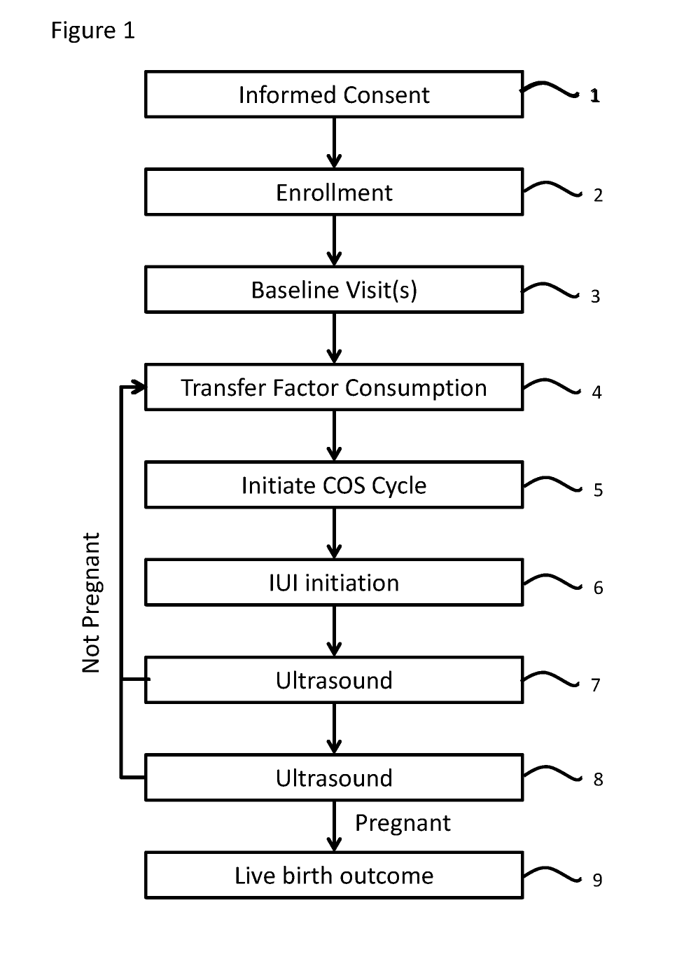

FIG. 1 is a flow diagram describing a COS/IUI test protocol modified with a transfer factor formulation, in accordance with embodiments disclosed herein. The initial step is an analysis. It begins with the subject signing a consent form and undergoing evaluation tests. The subject can be the woman, the man, or the couple seeking a child. Each participant is screened in the normal way for COS/IUI admission.

At block 1, informed consent is obtained. During this phase, general health indices (such as body mass index, weight, and medical history) are documented. Further at block 1, females diagnosed with tubular blockage are excluded because they cannot reasonably be helped with COS/IUI.

At block 2, subjects are selected for enrollment.

At block 3, optional baseline visits are conducted. In this phase urine, such as morning urine, and sperm samples are collected. The collected samples provide a background on which to measure change. The schedule is designed to provide consumption of transfer factor formulation for at least 30 days prior to COS, and nominally 45 days prior to IUI.

At block 4, transfer factor formulation consumption begins, for example beginning the first day of the next menstrual cycle after informed consent is obtained, and continuing for at least one full menstrual cycle. Consumption of transfer factor formulation is a core feature of the modified COS/IUI procedure. Typically, consumption begins at least 30 days before COS begins. Ideally, consumption would begin 100 days before COS. The reason is that 100 days allows both egg and sperm to develop fully in the presence of transfer factor formulation. Shorter periods of consumption still confer benefits to the COS/IUI procedure. In animal testing, as few as 3 days produced results in donor/recipient examples.

At block 5, COS is initiated. This provides time for fertility benefits to accrue. Blood tests, urine samples, sperm samples, and sonography are performed just prior to consumption. When sonography indicates the proper egg size and morphology, IUI is initiated. COS (controlled ovarian stimulation) begins nominally 3-5 days into the menses cycle, but could occur as late as 8 days to account for individual differences. COS timing may be modified by the laboratory tests, for example tests for E2, FSH, and LH. Trans-vaginal ultrasound and blood tests occur at this time. Morning urine collection may be included to study cortisol levels. Urine collection would not always be included into a modified COS/IUI procedure. For this specific protocol, transfer factor formulation consumption stops at IUI. Stopping is not a requirement. If consumption were continued, the health of the fetus would be expected to benefit.

At block 6, IUI begins. IUI typically begins 11-17 days into the menses cycle. For this specific embodiment, transfer factor formulation consumption is stopped for a woman. Stopping the woman's consumption at this time is not a technical consideration. It is a regulatory concern because of likely conception. The man may continue to consume in preparation for repeat procedures. IUI could be a clinical procedure. Alternatively, timed intercourse is considered a subset of IUI, and has roughly the same success rate.

At blocks 7 and 8, optionally ultrasound is used to detect pregnancy at 6 weeks (block 7) and at 12 weeks (block 8).

At block 9, pregnancy is achieved. If pregnancy is confirmed, live birth outcomes 9 are followed. If pregnancy is not confirmed, a repeated COS/IUI procedure 10 may be started.

If pregnancy is not achieved, the flow diagram may be repeated by returning to block 4. The number of repeats is not limited. In practice, three or four repeats are normally chosen as a maximum.

The protocols and timelines shown in FIGS. 1, 2, and 3, COS are effected with a chemical stimulant, such as Clomid® and Femara®. Chemically similar products are also within the scope of this disclosure. If the chemical is used to control ovulation timing, it is within this disclosure scope.

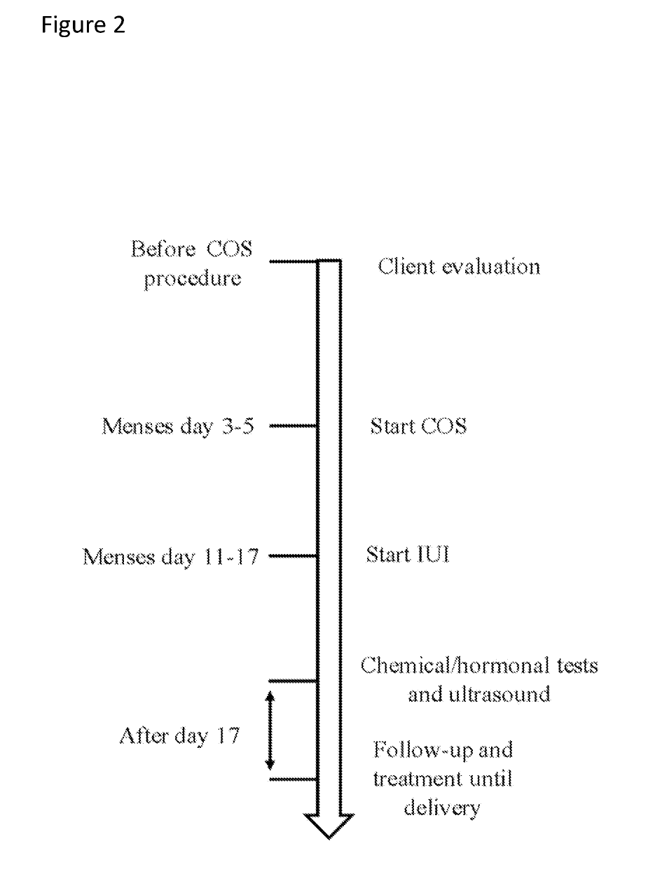

FIG. 2 shows a typical timeline for the standard COS/IUI procedure. The subject evaluation may occur shortly before COS. This is an unmodified procedure. There is no consumption of transfer factor formulation ahead of COS. COS typically begins 3-5 days into the menses cycle. Intra-uterine insemination (IUI) typically begins 11-17 days into the cycle. Tests for pregnancy (chemical, hormonal, ultrasound) begin after 17 days. Ultrasound at 6 weeks and 12 weeks is used to confirm and monitor pregnancy.

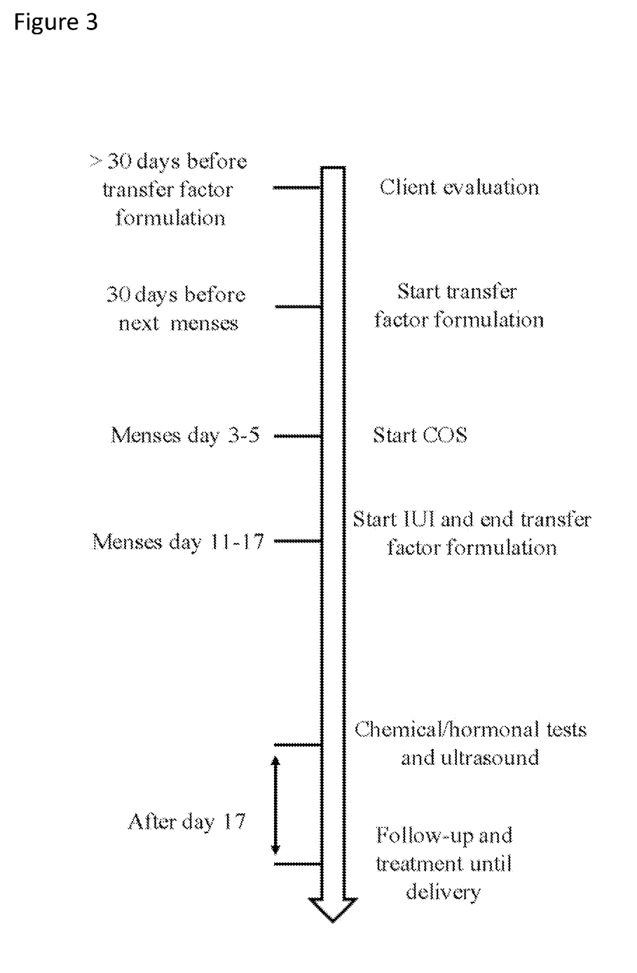

FIG. 3 shows an example timeline schedule for a typical COS/IUI with transfer factor formulation. Timing reflects estimates in FIG. 1. In FIG. 3, COS and IUI and follow-up testing maintain the same time spacing as shown in FIG. 2, but they are delayed by the introduction of transfer factor formulation. As shown in this example, consumption of transfer factor formulation begins at least 30 days before COS and extends to the beginning of IUI. The woman's consumption time period is nominally 40-45 days, which acknowledges that cycles are not always 28 days. The time period for consumption of transfer factor formulation before COS may vary. The “at least 30 days” is specific to this embodiment. The timing could be as little as 3 days before COS, or it could be 100 days (or more) before COS. Stress hormones and chemical markers may be affected with a mixture of transfer factor, lactic acid generating bacteria, and/or glucans. Examples include cortisol, alpha amalyase, adrenalin, and T4 measurements of thyroid function.

The below animal results are a very small fraction of the animal data available. This data supports the rationale behind administering transfer factor, lactic acid generating bacteria, and/or glucans to augment COS/IUI. Limited animal data is deemed sufficient because this instant application is focused on improving human COS/IUI. In particular, this current application is directed toward transfer factor formulation consumption before and during human COS/IUI.

### Example 3

A young bull nine months old was evaluated to have no live semen. The animal was then administered one ounce of transfer factor, lactic acid generating bacteria, and glucans daily for three months. After three months, 75 ampules of viable semen were collected from the animal. This is an above average yield.

### Example 4

Thirty-five (35) mature Hampshire ewes in Santa Rosa with fertility difficulty demonstrated conception at about 40% for several years. Administration of one ounce of the transfer factor formulation on Days 6 and 7 prior to breeding increased conception to about 95%.

### Example 5

A similar case was observed with donor cows. Without the transfer factor formulation, the best flush (donor) cow yielded 6 to 8 eggs; with usually only 1 or 2 eggs attaining Grade 1. With the transfer factor formulation, the same cow yielded 12 eggs; 10 of these were Grade 1. The food contained the same amount of transfer factor, lactic acid generating bacteria, and glucans as the instant transfer factor formulation.

### Example 6

In a commercial beef operation 100 cows were administered one ounce of fertility formulation 6 to 7 days before breeding. Conception improved by 30% with these protocols.

### Example 7

Cattle breeding without administration of transfer factor formulation yielded about 75% conception. Addition of the transfer factor formulation increased the rate of conception to 98%. Observations were consistent among herds.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

1. A method of improving the conception success rate of a human female subject undergoing a controlled ovarian stimulation and intrauterine insemination (COS/IUI) procedure, comprising:
administering to the female subject, prior to COS, a medical food formulation comprising an effective amount of transfer factor; and
continuing administration of the medical food formulation within the period between COS and IUI, wherein the COS/IUI conception rate obtained with the administration of the formulation is greater than the COS/IUI conception rate obtained without the administration of the formulation.

2. The method claim 1, wherein the effective amount of transfer factor is selected based on the female subject's body weight.

3. The method of claim 1, wherein the effective amount of transfer factor is between 0.05 mg and 50 mg per pound of human body weight.

4. The method of claim 1, further comprising testing the female subject for tubular blockage prior to COS.

5. The method of claim 1, further comprising testing the woman's male partner for sperm count prior to COS and/or prior to IUI.

6. The method of claim 1, wherein the administering occurs between five times per day and once per week.

7. The method of claim 1, wherein the administering begins from 3-100 days before the COS procedure.

8. The method of claim 1, further comprising administering an effective amount of glucans or polysaccharides.

9. The method of claim 8, wherein the glucans are derived from natural or hybrid mushrooms.

10. The method of claim 8, wherein an effective amount of glucans is between 0.1 mg and 10 mg per pound of human body weight.

11. The method of claim 8, wherein the transfer factor and glucans are separated and consumed at different times within a one week period.

12. The method of claim 1, further comprising administering an effective amount of lactic acid generating bacteria.

13. The method of claim 12, wherein an effective amount of lactic acid generating bacteria is between 0.47 mg and 10 mg per pound of human body weight.

14. The method of claim 12, wherein the transfer factor and lactic acid generating bacteria are separated and consumed at different times within a one week period.

15. The method of claim 1, further comprising administering a potassium supplement.

16. The method of claim 1, further comprising extending the administration until pregnancy is confirmed.

17. The method of claim 1, further comprising extending the administration through the entire pregnancy.

18. The method of claim 1, wherein the COS/IUI procedure with administration of the medical food formulation is repeated.

19. The method of claim 1, wherein the female subject's consumption of the medical food formulation ceases on or before the day of IUI.

20. The method of claim 1, further comprising confirming pregnancy by performing an ultrasound on the female subject.

21. The method of claim 1, further comprising administering to a male reproductive partner of the female subject the medical food formulation comprising the effective amount of transfer factor, wherein the effective amount is based on the body weight of the male.
